(19) United States
(12) Patent Application Publication
(10) Pub. No.: US 2017/0173263 A1
(43) Pub. Date: Jun. 22, 2017

(54) INJECTOR ASSEMBLY

(71) Applicant: New Injection Systems Ltd, Ipswich, Suffolk (GB)

(72) Inventor: Stephen Dunne, Ipswich, Suffolk (GB)

(21) Appl. No.: 15/118,241

(22) PCT Filed: Feb. 12, 2015
(86) PCT No.: PCT/GB2015/050393

§ 371 Date: Aug. 11, 2016

(30) Foreign Application Priority Data
Feb. 12, 2014 (GB) 1402423.6

(51) Int. Cl. A61M 5/20; A61M 5/28; A61M 5/145

(52) U.S. Cl. CPC A61M 5/2033; A61M 5/1454; A61M 5/28; A61M 2205/582; A61M 2205/581; A61M 5/36

(57) ABSTRACT

An injector assembly comprises a collapsible container for containing a liquid medicament (15); and pressurising means (21, 101) arranged to apply a first force to the collapsible container to continuously pressurise the liquid medicament 15 during storage of the liquid medicament 15. The pressurising means (21, 101) is actuatable to apply a second force, which is greater than the first force, to the collapsible container.

The invention relates to an injector assembly, for example an injector assembly that may be used by a patient for self-injection or by a medical professional.

### BACKGROUND

Pre-filled containers for use in an injector assembly, such as pre-filled syringes and pre-filled cartridges, are filled by manufacturers in controlled sterile environments, eliminating the need for a patient or a medical professional to fill them from vials or ampoules prior to use. Pre-filled syringes typically have a storage life of two years or more.

Auto-injectors comprising pre-filled syringes and cartridges provide automation of the injection stroke, alleviating the need for the patient or medical professional to actuate a plunger rod to deliver medication.

Industry standard ‘staked needle’ pre-filled syringes, such as the BD Hypak, the Gerresheimer RTF or ClearJect, the Schott TopPak, the Daikyo Crystal Zenith® Syringe, and other commercially available glass or plastic ready to fill syringes, are commonly used as the primary pack or primary container for auto-injectors.

Historically the industry has been reliant upon these well-established off-the-shelf primary containers, usually the glass versions. Most of the alternative auto-injector technologies require a bespoke primary container, which introduces unwanted risk and cost to the development process. However, the standard glass pre-filled syringe and to a lesser extent the glass cartridge, present a number of problems. In particular, glass pre-filled syringes and glass cartridges are fragile and not well suited to use in spring-driven auto-injector devices.

Various patents describe devices that try to overcome some of the problems associated with the use of pre-filled syringes. Most of the prior art attempts to solve such problems involve the use of a completely new primary pack which the industry is reluctant to use. The pharmaceutical industry is reluctant to adopt entirely new designs of packaging.

A trend in the industry is that so called small molecule drugs, or conventional drugs, are being replaced by large molecule biopharmaceutical (biological) drugs. This trend results in further criteria that need to be considered when designing a primary pack or an injector.

The vast majority of biological drugs have to be administered parenterally. Most protein drug formulations are destroyed by digestive enzymes if taken orally, and it is difficult to get sufficient active dosage to transfer across a mucous membrane or epithelium, so the bioavailability is typically low. All antibody drugs are likely to be for parenteral administration for the foreseeable future. Most injections and infusions have never been particularly popular with the recipient. They hurt, or at least they are perceived to hurt especially if the needles used for their delivery are of large diameter.

Many biological drugs are more viscous than small molecule drugs. That makes them more difficult to inject, as either larger diameter needles are required to minimize flow resistance, or much higher pressures are required if the more favoured small diameter needles are used. Higher delivery pressures can lead to breakage of containers, which is more of a problem when glass containers are used. One possible solution is to dilute the highly viscous drug, thereby reducing its viscosity. If this is done, however, the total volume can exceed the maximum acceptable injectable amount of about 1.5 ml. To inject larger volumes in excess of 1.5 ml, patch or bolus pumps need to be used.

WO2014/080020 discloses an auto-injector assembly. The disclosure in this document is incorporated by reference in its entirety.

The auto-injector assembly of WO2014/080020 comprises a medicament container defining a substantially cylindrical chamber containing a liquid medicament. The medicament container may be, for example, a cartridge or a syringe. A biasing means acts to continuously pressurise the liquid medicament. The assembly further comprises a hypodermic needle for parenteral administration of the liquid medicament, and a removable needle cap for maintaining the hypodermic needle in sterile conditions until use. Means for establishing fluid communication between the medicament container and the hypodermic needle is also provided, such that the pressurised liquid medicament is automatically delivered through the hypodermic needle when such fluid communication has been established.

The injector assembly of WO2014/080020 allows for the use of industry standard injection cartridges or ready to fill syringes (pre-filled syringes) as medicament containers to create auto-injectors and bolus pumps (large volume injectors) that have dry needles during storage and various other advantages. Both plastic and glass versions of standard injection cartridges and ready to fill syringes are available from a number of suppliers including Gerresheimer, Schott and Becton Dickinson.

Glass injection cartridge dimensions and tolerances are defined in International Standard ISO 13926-1. Stoppers and seals (cap and disc) are described in International Standard ISO 13926-2 and 3. Ready to fill syringes or pre-filled syringes dimensions and tolerances are defined in ISO 11040-4.

One potential problem with the device described in WO2014/080020 is that for very viscous drugs the pressure required for realistic injection times of under 10 seconds needs to be relatively high. The development of such high pressures can cause safety issues and problems with integrity of the assembly during storage. For instance the stopper or piston may leak. Or the elastomer cap may leak over prolonged storage.

### DESCRIPTION OF THE INVENTION

The invention provides an injector assembly, and a method of using an injector assembly, as defined in the appended independent claims, to which reference should now be made. Preferred or advantageous features of the invention are set out in dependent sub-claims.

Thus, an injector assembly may comprise a collapsible container for containing a liquid medicament; and pressurising means arranged to apply a first force to the collapsible container to continuously pressurise the liquid medicament contained therein during storage of the liquid medicament. The pressurising means is actuatable to apply a second force to the collapsible container. The second force is greater in magnitude than the first force.

The first force applies a continuous pressure to a liquid medicament contained within the collapsible container, which allows the liquid medicament to be stored under pressure. There are a number of benefits provided by storage of a liquid medicament under a continuous pressure. For example, storage of a liquid medicament under pressure advantageously improves leak detection. If the primary container or a closure of the container is damaged, the positive storage pressure causes the medicament to leak thereby alerting the user to a problem. The positive pressure in the container during storage also minimizes the risk of drug contamination and loss of sterility.

The injector assembly may advantageously comprise the liquid medicament. That is, it may be advantageous that the collapsible container of the injector assembly be filled with a liquid medicament and the liquid medicament be retained under pressure due to the action of the first force on the collapsible container.

The fact that the pressurising means can apply a second force to the collapsible container, which is greater than the first force, allows the injector assembly to be formed from fragile materials such as glass yet still be used for high pressure delivery of liquid medicaments. High pressure delivery is useful for reducing the delivery time required for viscous medicaments, such as biological medicaments, whilst still allowing small gauge needles to be used. As a low storage pressure is used, the benefits of storing the medicament under positive pressure may be achieved. The hazards of pressurising a fragile glass container and storing and shipping a highly pressurised container may be avoided. As a relatively high delivery pressure can subsequently be applied to the collapsible container when required, the benefits of high pressure delivery may be achieved, which may be particularly advantageous in the context of delivering highly viscous medicaments. The second higher force need not be applied to the collapsible container for any significant length of time, thereby reducing hazards associated with pressurising the container.

The pressurising means may comprise a first biasing means and a second biasing means. Thus, the first force may be exerted by the first biasing means and the second force may be exerted by the second biasing means. It may be preferred that the first biasing means is a first spring, for example a first helical spring, and/or the second biasing means is a second spring, for example a second helical spring. Other biasing means may be envisaged. For example, the first force and/or the second force may be provided by gas pressure.

The injector assembly preferably comprises a housing locating the collapsible container and the pressurising means, and the pressurising means is retained within the housing such that it acts on the collapsible container. Thus, a first biasing means may be retained or located within the housing such that it continually acts on the collapsible chamber until and/or after the pressurising means is actuated. A second biasing means may be retained or located within the housing such that it acts on the collapsible chamber after the pressurising means has been actuated. The first biasing means and the second biasing means may be arranged within the housing in parallel or in series. If the first and second biasing means are springs, it is preferred that the second biasing means is a spring having a higher compression force than the first biasing means.

Preferably, when the pressurising means is in an unactuated condition the second biasing means is restrained within the injector assembly such that it does not exert a force on the collapsible chamber. Actuation of the pressurising means may then cause the second biasing means to exert a force resulting in the second force being applied to the collapsible chamber.

The pressurising means may comprise only a single biasing means, the injector assembly being arranged such that both the first force and the second force can be exerted by the single biasing means. For example, the single biasing means may be a single helical spring. For example, when the pressurising means is in an unactuated condition a first portion of the single biasing means may be restrained within the assembly such that only a second portion of the single biasing means exerts the first force on the collapsible chamber. Actuation of the pressurising means may then allow both first and second portions of the single biasing means to exert the second force on the collapsible chamber. If the single biasing means is a helical spring having, for example, eight coils, the spring may be restrained by a restraining means such that only one or two coils of the spring act on the collapsible container. The one or two coils of the spring exert the first force. Actuating the pressurising means by removal of the restraining means allows all eight of the coils of the spring act on the collapsible container thereby exerting the second force that is greater than the first force.

The pressurising means may be actuated to apply the second force to the collapsible container automatically or manually. For example, the pressurising means may be actuated to apply the second force to the collapsible container by depressing a button. The button may automatically actuate the pressurising means when the injector is used. Alternatively, the pressurising means may be actuated by removing a cap or a latch.

The injector assembly may comprise a releasable retaining means which can releasably retain the pressurising means. The pressurising means may be actuated by release of the retaining means. The releasable retaining means may comprise a retaining arm, which at one end attaches to the pressurising means and at the other end releasably attaches to the injector assembly, for example to a cap at the proximal end of the injector assembly. In one embodiment, the releasable retaining means comprises a catch, e.g. a hook, that is able to releasably engage with the cap, wherein the cap comprises a hole that allows passage of the catch upon displacement of the hook from the cap.

The injector assembly may comprise a liquid medicament, for example a liquid biological medicament. That is, the injector assembly may be pre-filled with a liquid medicament contained within the collapsible container. The first force may act to continuously pressurise the liquid medicament to a pressure that is greater than atmospheric pressure but lower than 10 bars, preferably less than 5 bars. The pressure in the liquid medicament may be less than 10 bars, 9 bars, 8 bars, 7 bars, 6 bars, 5 bars, 4 bars, 3 bars, or 2 bars when the first force is applied to the collapsible container.

The second force may act to continuously pressurise the liquid medicament to a pressure that is greater than the first pressure and higher than 5 bars, preferably higher than 10 bars. The pressure in the liquid medicament may be higher than 6 bars, 7 bars, 8 bars, 9 bars, 10 bars, 12 bars, 15 bars, or 20 bars when the second force is applied to the collapsible container.

The fact that the pressure in the liquid medicament during storage is lower relative to a delivery pressure reduces the stresses in the injector assembly, thereby increasing the safety and shelf-life of the injector assembly.

The injector assembly may be transported and stored with a liquid medicament within the collapsible container but without a delivery means coupled to the injector. The injector assembly may be coupled to a delivery means immediately prior to use. Alternatively, the injector assembly may comprise a delivery means for delivering the liquid medicament to the patient. For example, the injector assembly may comprise a hypodermic needle arranged to deliver the liquid medicament from the collapsible chamber.

An injector assembly may further comprise a shield for the delivery means, for example a needle shield. A linkage system may be provided, wherein said linkage system automatically actuates the pressurising means when the shield is retracted or removed. Thus, the injector assembly may be made ready for delivery when the shield is retracted or removed.

The collapsible container may be defined by rigid internal walls and a piston that is slidably arranged within the rigid internal walls, the volume of the collapsible container varying depending on the position of the piston. In such a configuration, the pressurising means applies the first force and the second force to the piston to pressurise a liquid medicament contained within the collapsible container. The collapsible container may be a bellows. The collapsible container may include both a bellows and a piston, for example a bellows may provide a lining to a collapsible container defined by rigid walls and a piston.

It is preferred that the collapsible container is closed by a closure and that the liquid medicament is delivered when the closure is opened or breached. For example, an opening to the collapsible container may be closed by a pierceable septum. The septum may be pierced by a hollow needle or cannula that is in fluid communication with a delivery means such as a hypodermic needle. When the septum is pierced the force applied to the collapsible container may force the liquid medicament out of the collapsible container. The collapsible container may be closed by a normally-closed valve, such as an aerosol valve. Opening of the normally-closed valve may allow passage of the liquid medicament from the container.

The collapsible container is preferably defined within a standard glass pre-filled syringe or glass container. The injector assembly may advantageously be a component of an auto-injector, or may be an auto-injector, for example a hand-held auto-injector.

In a preferred embodiment, an injector assembly comprises a glass container housing a piston and a first spring and a second spring arranged to act on the piston. A storage pressure is applied to the piston by the first spring and a delivery force is provided to the piston by the second spring. The assembly includes a locking and release mechanism for the second spring or main power spring. In this way during storage the collapsible container is kept at a lower than injection pressure to minimize stresses in the assembly.

In a preferred embodiment, the injector assembly is an auto-injector assembly, which comprises a medicament container defining a substantially cylindrical chamber containing a liquid medicament, a proximal end of the chamber being closed by a piston slidably located within the cylindrical chamber, and a distal end of the chamber being closed by a container seal spanning an opening at a distal end of the medicament container; pressurising means arranged to apply a first force to the piston and acting to bias the piston towards the container seal, thereby continuously pressurising the liquid medicament during storage of the liquid medicament, wherein the pressurising means is actuatable to apply a second force to the collapsible container, the second force being greater than the first force; a hypodermic needle for parenteral administration of the liquid medicament, a removable needle cap for maintaining the hypodermic needle in sterile conditions until use, and means for establishing fluid communication between the chamber and the hypodermic needle such that the pressurised liquid medicament is automatically delivered through the hypodermic needle when communication has been established.

The present invention may be used in conjunction with any drug whether a solution or a suspension or a mixture of these of any viscosity and density. Any of the drugs listed below, either on its own or a mixture thereof, may be injected using an injector assembly as disclosed herein:

17-alpha hydroxyprogesterone caproate, Corticotropin (ACTH), Laronidase, Factor VIII, Von Willebrand Factor Complex, Alefacept, Apomorphine Hydrochloride, Darbepoetin Alfa, Nelarabine, Bevacizumab, Interferon beta-1a, 11 mcg, Interferon beta-1a, 33 mcg, Factor IX complex, Interferon beta-1b, Ibandronate Sodium, Botulinum Toxin, Protein C Concentrate, Alglucerase, Imiglucerase, Injection, Secretin, Synthetic, Human, 1 Microgram, Glatiramer actate, Decitabine, Desmopressin acetate, Idursulfase, Etanercept, Epoetin alfa, Anadalufungin, Cetuximab, Ethanolamine Oleate, Hyaluronic acid derivatives, Agalsidase beta, Factor IX non-recombinant, Factor IX recombinant, Factor VIII (human), Factor VIII (porcine), Factor VIII recombinant, Feiba VH, Immune globulin (intravenous) (IVIG), Enfuvirtide, Immune globulin (intravenous) (IVIG), Somatropin, Hepatitis B Immune, Globulin (intravenous) (IVIG), Trastuzumab, von Willebrand factor complex, Adalimumab, Insulin for administration through DME (i.e., insulin pump), Hyaluronic acid derivatives, Mecasermin, Gefitinib, Levoleucovorin calcium, Ranibizumab Injection, Pegaptnib, Urofollitropin, Micafungin, Botulinum toxin type B, Aglucosidase alfa, Galsulfase, Somatropin, Factor VIIa, Atacept, Hyaluronic acid derivatives, Hyaluronan derivative, Immune globulin (intravenous) (IVIG), Hemin, Peginterferon alfa-2a, Peginterferon alfa-2b, Epoetin alfa, Somatrem, Efalizumab, Interferon beta-1a, subq, Zoledronic Acid, Infliximab, Treprostinil, Fluocinolone acetonide, intravitreal implant, Zidovudine, Eculizumab, Lanreotide, Histrelin implant, Palivizumab, Hyaluronic acid derivatives, Temozolomide, Antithrombin III (Human), Natalizumab, Panitumumab, Immune globulin (intravenous) (IVIG), Azacitidine, Verteporfin, Hyaluronidase, Bovine, Preservative Free, Naltrexone Depot, Teniposide, Omalizumab, 90Y-Ibritumomab tiuxetan, ADEPT, Aldesleukin, Alemtuzumab, Bevacizumab, Bortezomib, Cetuximab, Dasatinib, Erlotinib, Gefitinib, Gemtuzumab, Imatinib, Interferon alpha, Interleukin-2, Iodine 131 tositumomab, Lapatinib, Lenalidomide, Panitumumab, Rituximab, Sorafenib, Sunitinib, Thalidomide, Trastuzumab.

The injector assembly may also be used to deliver biologics or small molecule drugs including a wide range of medicinal products such as vaccines, blood and blood components, allergenics, somatic cells, gene therapy, tissues, and recombinant therapeutic proteins, and substances that are (nearly) identical to the body's own key signalling proteins may also be injected using the invention. Examples are the blood-production stimulating protein erythropoetin, or the growth-stimulating hormone named (simply) “growth hormone” or biosynthetic human insulin and its analogues.

The injector assembly may also be used to deliver monoclonal antibodies. These are similar to the antibodies that the human immune system uses to fight off bacteria and viruses, but they are “custom-designed” (using hybridoma technology or other methods) and can therefore be made specifically to counteract or block any given substance in the body, or to target any specific cell type.

The injector assembly may also be used to deliver receptor constructs (fusion proteins), usually based on a naturally-occurring receptor linked to the immunoglobulin frame. In this case, the receptor provides the construct with detailed specificity, whereas the immunoglobulin-structure imparts stability and other useful features in terms of pharmacology.

The injector assembly may also be used to deliver any of the following: Alpha1-Adrenergic Antagonists, Analgesic Agents, Anesthetics, Angiotensin Antagonists, Inflammatory Agents, Antiarrhythmics, Anticholinergics, Anticoagulants, Anticonvulsants, Antidiarrheal Agents, Antineoplastics and Antimetabolites, Antineoplastics and Antimetabolites, Antiplasticity Agents, Beta-Adrenergic Antagonists, Bisphosphonates, Bronchodilators, Cardiac Inotropes, Cardiovascular Agents Central Acting Alpha2-stimulants, Contrast Agents, Converting Enzyme Inhibitors, Dermatologics, Diuretics, Drugs for Erectile Dysfunction, Drugs of Abuse, Endothelin Antegonists, Hormonal Agents and Cytokines, Hypoglycemic Agents Hypouricemic Agents and Drugs Used For Gout, Immunosuppressants, Lipid Lowering Agents, Psychotherapeutic Agents, Renin Inhibitors, Serotonergic Antagonist Steroids, Sympathomimetics, Thyroid and Antithyroid Agents, Vasodilators, Vasopeptidase Inhibitor.

The injector assembly may also be used to deliver any drug with indications for Rheumatoid arthritis or Multiple sclerosis, Hemophilia A or B, Vasculitis, Beta-thalassemia, Anemia, blood coagulation disorders, Von Willebrand disease, Sickle cell anemia, Solid Tumours, Leukemia, all cancers including liver, bladder, renal, esophageal, ovarian, breast, prostate, pancreatic, colorectal or lung, malignant melanoma, multiple myeloma, crohn's disease, ulcerative colitis, uveitisfabry disease, pompe disease, viral infections, HIV, Hepatitis A, B, C, Marburg virus, Wolman disease, Muscular dystrophy, botulism, muscular diseases, ebola virus, gout, acne, psoriasis, COPD, asthma, Alzheimer's, ALS, migraine, synovitis, fibrosis or any other indication.

The injector assembly may also be used to deliver any drug approved and listed by the FDA in the USA or any other national or international agency. Additionally any generic or biosimilar or biobetter drug on the market or in development.

The injector assembly may also be used to deliver any one of the following: Lipitor, a cholesterol-lowering statin drug, Nexium, an antacid drug, Plavix, a blood thinner, Advair, Abilify, an antipsychotic drug, Seroquel, an antipsychotic drug, Singulair, an asthma drug; Crestor, a cholesterol-lowering statin drug, Actos, a diabetes drug or Epogen, an injectable anemia drug.

The injector assembly may also be used to deliver any other drug not listed above capable of being injected and available at present or being developed by any pharmaceutical company or any other company anywhere in the world.

The invention may be used to inject humans or animals.

A method of using an injector assembly comprising a collapsible container containing a liquid medicament; and pressurising means arranged to apply a first force to the collapsible container to continuously pressurise a liquid medicament contained therein during storage may comprise the steps of: (a) actuating the pressurising means to apply a second force to the collapsible container, the second force being greater than the first force, and (b) establishing communication between the collapsible container and a delivery means for delivering the liquid medicament to the patient, the second force acting to eject the liquid medicament from the collapsible container through the delivery means.

The pressurising means may be actuated simultaneously with or before communication is established between the collapsible container and the delivery means. Alternatively, communication may be established between the collapsible container and the delivery means before the pressurising means is actuated. The injector assembly may be any injector assembly described above.

Specific embodiments of the invention will now be described with reference to the figures, in which:

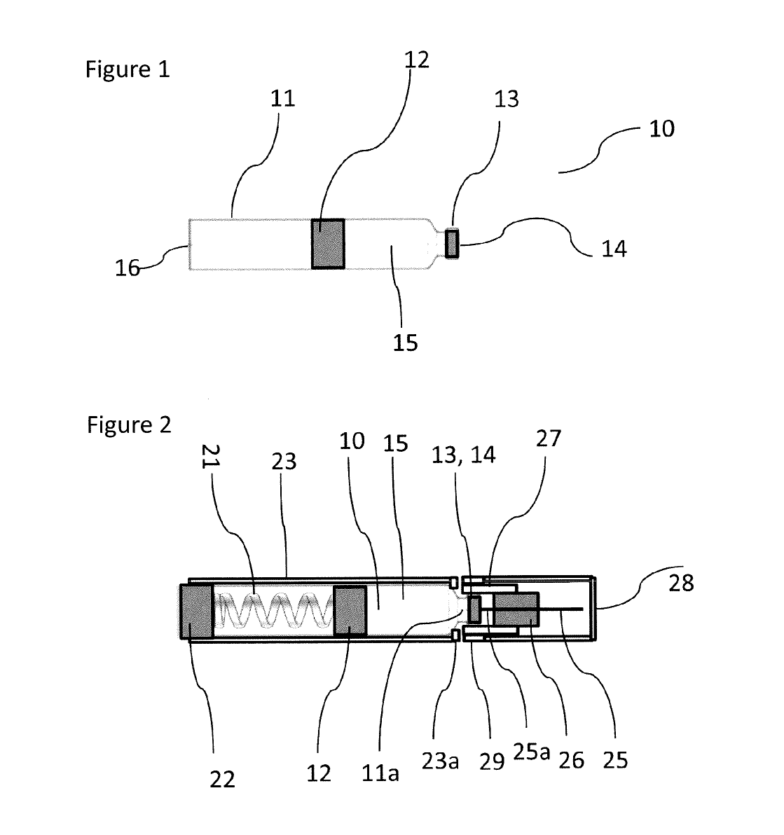

FIG. 1 is a schematic illustration of a standard pre-filled cartridge as typically used in the pharmaceutical industry, and suitable for use as a medicament container in an auto-injector assembly according to the invention.

FIG. 2 is a schematic illustration of an auto-injector assembly according to the prior disclosed in WO2014/080020.

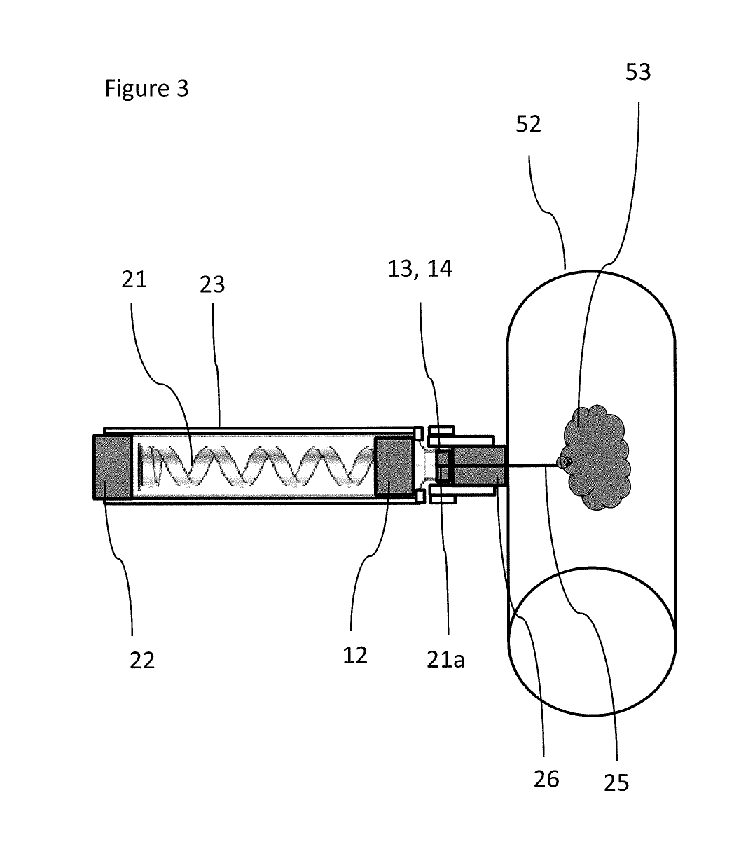

FIG. 3 illustrates the use of the auto-injector assembly of FIG. 2.

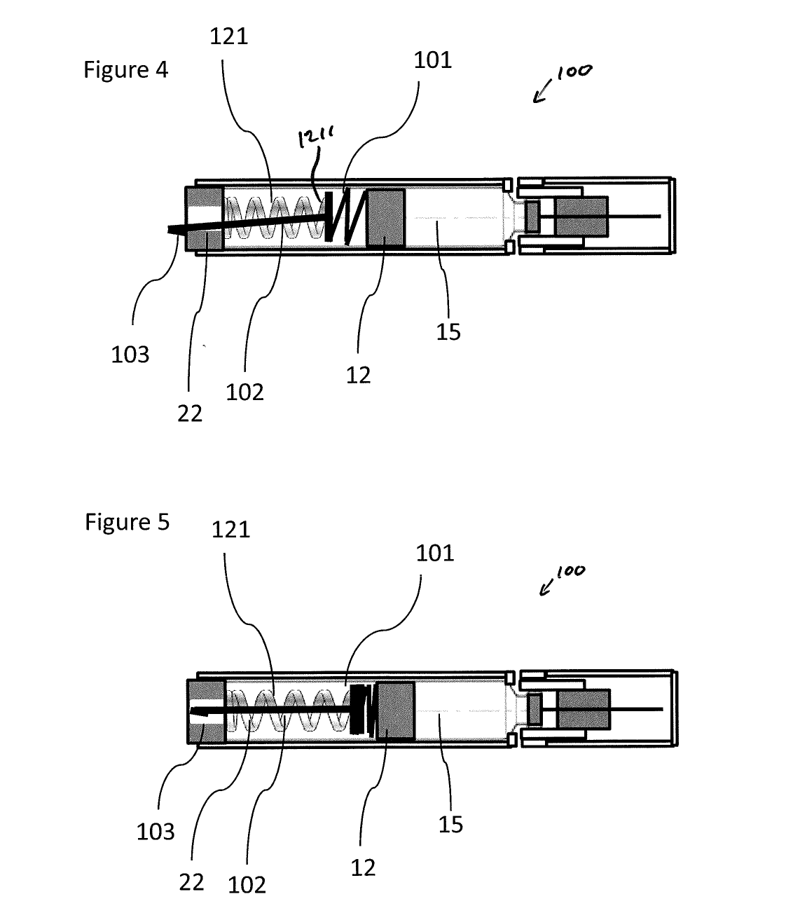

FIG. 4 is a schematic illustration of an auto-injector assembly according to the present invention.

FIG. 5 is a schematic illustration of an injector assembly after actuation of a pressurising means, but prior to release of the liquid medicament from the medicament container.

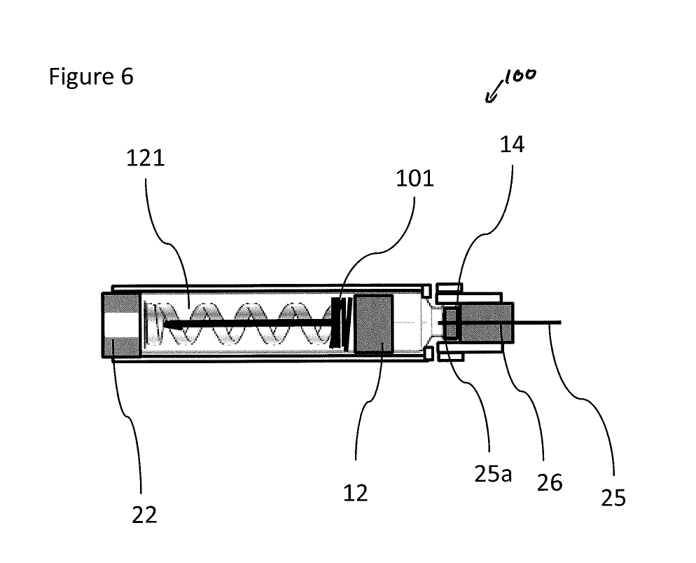

FIG. 6 is a schematic illustration of an auto-injector assembly according to the present invention during use (injection).

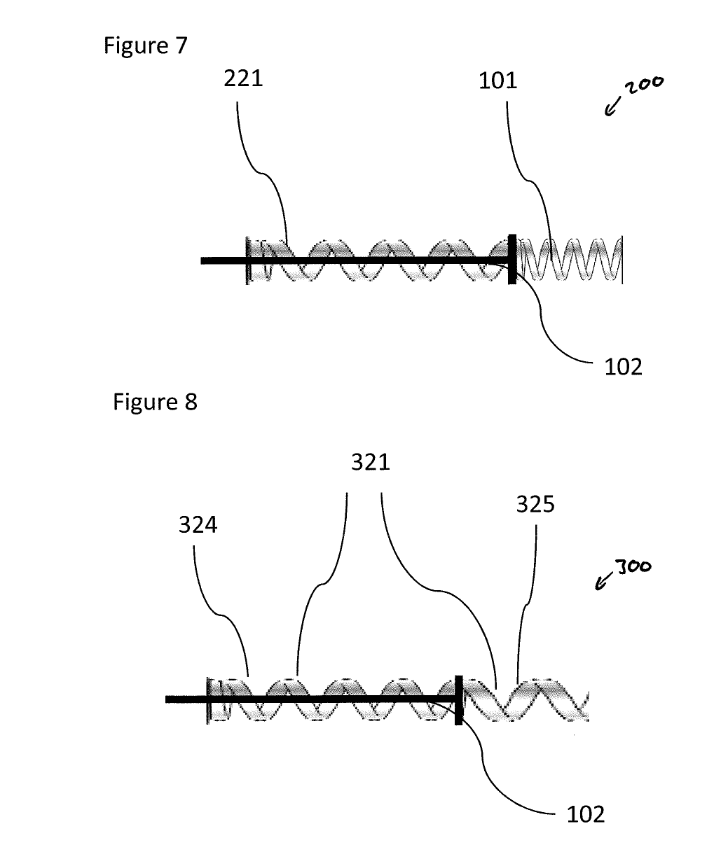

FIGS. 7 and 8 show alternative spring arrangements suitable for use as pressurising means in an injector assembly according to one or more embodiments of the invention.

FIG. 1 illustrates a standard cartridge 10 used in the pharmaceutical industry, for example for dental injections and insulin injections. A cartridge body or barrel 11 has a liquid drug solution or suspension 15 within. The liquid 15 is contained within a cartridge chamber defined by a movable piston or stopper 12 at one end (towards a proximal end of the cartridge) and a container seal in the form of a rubber seal or septum 14 held into place by a metal crimp 13 at the other end (a distal end of the cartridge). The barrel 11 is open at its proximal end 16. The cartridge 10 may act as the primary pack in an auto-injector assembly according to an embodiment of the invention.

FIG. 2 shows the use of the cartridge 10 of FIG. 1 as a primary container in an auto-injector assembly according to prior art patent application WO2014/080020. The assembly has a helical spring 21 acting to pressurise the liquid contents 15 of the cartridge 10 by urging the stopper 12 forward in a direction towards the rubber septum 14. The spring 21 is located by a spring lock or cap 22, which in turn is held into place by a casing 23. The casing 23 has lugs 23a which are engaged with the cartridge neck 11a.

The casing 23 has viewing holes (not shown) in order to inspect the drug before injection.

A valve housing 27 is sealed against a radially external portion of the metal crimp 13. The valve housing locates a slidable shuttle 26, itself locating two needles that are in fluid communication with each other. A hypodermic needle 25 extends from a distal end of the shuttle 26 for injecting the patient, and a hollow needle 25a extends from a proximal end of the shuttle 26 for perforating the septum 14. In use, the shuttle 26 is held against the patient's skin after inserting the hypodermic needle 25 into the patient. The shuttle slides within the valve housing and the hollow needle 25a is forced through the septum 14. This action results in establishment of fluid communication between the cartridge chamber and the hypodermic needle 25.

Because the liquid contents 15 of the chamber are pressurised, the liquid flows into the patient via the needles 25a and 25.

The needles 25a and 25 may be formed as a single double-ended needle or as separate needles connected by a channel defined through the shuttle 26.

A sterile removable needle cap 28 keeps the hypodermic needle 25 sterile before use for injection.

The spring lock 22 may form an oxygen and humidity barrier, in which case the stopper 12 need not be itself an oxygen and humidity barrier. This may allow for a greater choice of materials for the stopper, including self lubricating materials such as PTFE and Silicone. Self lubricating materials may eliminate or reduce the requirement to lubricate the internal surfaces of the cartridge to enable the stopper to slide.

Since the spring lock 22 is not in contact with the drug, a wide range of barrier materials can be used in its manufacture. Barrier materials may be available that were not previously available for use as a stopper, due to contact between the stopper and the drug.

The spring lock 22 may be held in place by an outer casing or any other means such as a flange formed as part of the syringe or cartridge barrel.

In the injector assembly of FIG. 2, the liquid drug is stored in an unmodified standard cartridge. This provides the advantage that no new stability trials are needed with existing drugs. There may be a limit to the delivery pressure that can be provided by the spring, however, as it is undesirable to store or transport the assembly when the liquid drug is pressurized at high pressures.

FIG. 3 illustrates use of the prior art auto-injector assembly of FIG. 2. The hypodermic needle 25 has penetrated an injection site 52. The hollow needle 25a has pieced the septum 13 and the stopper 12 has pushed the liquid drug contents out of the cartridge under the action of the spring 21. The liquid medicament contents have been delivered 53. The spring 21 is now extended and the stopper has been moved to a distal end of the cartridge.

FIGS. 4-7 illustrate injector assemblies according to embodiments of the present invention that allow high pressure delivery of liquid medicaments. This reduces the delivery time required for viscous liquid medicaments, such as biological medicaments, whilst allowing small gauge needles to be used. The liquid medicaments contained within these injector assemblies are stored under pressure at relatively low pressures compared to the delivery pressure, which reduces the stresses in the injector assemblies, thereby increasing the shelf-life of the injector assemblies.

FIG. 4 illustrates an auto-injector assembly according to an embodiment of the present invention. The auto-injector assembly is shown in its storage state. As in the injector illustrated in FIG. 1, a liquid medicament 15 is contained in a collapsible container, which is defined by rigid internal walls and a piston that is slidably arranged within the rigid internal walls. Components of the injector assembly that are the same as described above in relation to FIGS. 1 to 3 have been given the same reference numerals and function in the same way. The injector assembly of FIG. 4 differs in the pressurising means used to pressurise the liquid medicament 15 for storage and delivery.

The injector 100 comprises a pressurising means for applying forces to a piston 12, thereby pressurising the liquid medicament 15. The pressurising means comprises a first helical spring 101 and a second helical spring 121. A retaining arm 103 passes through a hole in cap 22 and has at its proximal end a catch 103 that hooks onto the end of the cap 22 in order to hold the retaining arm in place. The retaining arm also has a T-bar at its distal end, which acts to restrain movement of one end 1211 of the second spring 121 such that the second spring 121 does not directly exert a force on the piston 12 while it is restrained. The first spring 101 acts between the restrained second spring 121 and the piston 12 to apply a first force to the piston. The first spring 101 has a lower compression force than the second spring 121. The retaining arm 102 and the catch 103 hold the second spring 121 in a compressed state, and in such a way that the first spring 101 is compressed and provides a biasing force between the second spring 121 and the piston 12. This biasing force maintains the liquid contents 15 of the injector in a pressurized state, but at a lower pressure that would be generated by the second spring 121 were the second spring to be acting directly between the cap 22 and the piston 12. The catch 103 is releasably held by the cap 22.

The first spring 101 can be sized to keep the liquid contents 15 at a desired pressure during storage independently from the pressure required for injection. By restraining the second spring 121, and by using the first spring 101 to pressurise the liquid during storage, the liquid medicament 15 in the cartridge 10 may be maintained at a lower pressure than that generated by the second spring 121 during injection. In some cases, for instance where very viscous drugs are concerned, this provides an advantage over the injector described in WO2014/080020 where the liquid contents 15 are kept under the full pressure generated by spring 21 during storage.

FIG. 5 shows the injector assembly after the catch 103 has been released, but prior to release of the liquid medicament 15 from the collapsible container. Catch 103 is released from cap 22 by moving the catch 103 laterally, so that the catch unhooks from the end of the cap 22, and passes through the hole in cap 22. Release of the catch 103 releases the second spring 121 and allows it to compress the first spring 101 and apply a force to the piston 12. The first spring 101 may be fully compressed. The second spring 121 thereby applies a force to the piston and to the collapsible container, which is larger than the force applied by the first spring 101 prior to release of the catch 103. This larger force urges the piston or stopper 12 towards a distal end of the medicament container and increases the pressure of the liquid medicament.

In FIG. 6 the auto-injector is shown during its injection state. The catch 103 has been released, the first spring 101 has been compressed by the second spring 121 and the needle 25 has perforated the closing seal or septum 14. The liquid contents are delivered through the needle by the force applied to the collapsible container by the second spring.

In some embodiments, the catch or latch 103 may be connected to a button for release. It may be released after or before the needle 25 has perforated the septum 14. A linkage system may be employed to release catch 103 when a safety needle shield has been retracted or if a cap has been removed.

FIG. 7 illustrates one embodiment of a pressurising means 200 for use in an injector assembly as described herein. In this embodiment a first spring, or pressurising spring 101 is different and separate to a second spring, or injection spring 121. This is the embodiment of a pressurising means illustrated in the injector of FIGS. 4, 5, and 6.

FIG. 8 shows an alternative embodiment of a pressurising means 300 for use in an injector assembly as described herein. The pressurising means 300 of FIG. 8 comprises a single spring 321 in place of the two springs employed in the pressurising means 200 of FIG. 7. A retaining arm 302 holds the single spring 321 at a mid position to restrain some of the coils of the single spring. In doing so, a first portion 325 of the single spring 321 can exert a force on a collapsible chamber of an injector, whereas coils from a second portion 324 of the single spring 321 are prevented from exerting a force on the collapsible container. The first portion 125 of the single spring exerts a lower force than would be exerted by the entire unrestrained single spring. In particular, the first portion 125 will act to continuously pressurise a liquid medicament 15 at a lower pressure than will be generated by the entire single spring 321 when the retaining arm 102 is released. When the retaining arm 102 is released, all of the coils of the single spring can act to apply a delivery force to the collapsible container.

In some embodiments, the pressurising means may include any means that is able to apply a first continuous force to the collapsible container and which is actuatable to apply a second force to the collapsible container, wherein the second force is greater than the first force. The first continuous force should be able to be maintained continuously for extended periods of time, for example for several years. Such pressurising means includes mechanical pressurising means, such as springs, as well as liquids and gases. Different pressurising means may be used in combination. For example, the pressurising means may comprise a first biasing means and a second biasing means, wherein the first biasing means is a spring and the second pressurising means is a gas, or vice versa.

In some embodiments, the injector assembly may comprise a needle or it may be a needleless injector assembly. For example, the injector assembly may use a needleless transdermal delivery system.

1. An injector assembly comprising:
a collapsible container for containing a liquid medicament; and
pressurising means arranged to apply a first force to the collapsible container to continuously pressurise a liquid medicament contained therein during storage of the liquid medicament;
wherein the pressurising means is actuatable to apply a second force to the collapsible container, the second force being greater than the first force,
wherein the pressurising means comprises a first biasing means and a second biasing means, wherein the first force is exerted by the first biasing means and the second force is exerted by the second biasing means; and
wherein the first biasing means is retained or located within the housing such that it continually acts on the collapsible chamber until and/or after the pressurising means is actuated.

2. An injector assembly according to claim 1, in which the first biasing means is a first spring and the second biasing means is a second spring.

3. An injector assembly according to claim 1 or claim 2 in which, when the pressurising means is in an unactuated condition the second biasing means is restrained within the assembly such that it does not exert a force on the collapsible chamber, actuation of the pressurising means causing the second biasing means to exert a force resulting in the second force being applied to the collapsible chamber.

4. An injector assembly comprising:
a collapsible container for containing a liquid medicament; and
pressurising means arranged to apply a first force to the collapsible container to continuously pressurise a liquid medicament contained therein during storage of the liquid medicament;
wherein the pressurising means is actuatable to apply a second force to the collapsible container, the second force being greater than the first force;
wherein the pressurising means comprises a single biasing means, the assembly being arranged such that both the first force and the second force can be exerted by the single biasing means;
in which, when the pressurising means is in an unactuated condition a first portion of the single biasing means is restrained within the assembly such that only a second portion of the single biasing means exerts the first force on the collapsible chamber, actuation of the pressurising means causing both first and second portions of the single biasing means to exert the second force on the collapsible chamber.

5. An injector assembly according to claim 4, wherein the single biasing means is a helical spring.

6. An injector assembly according to any of the preceding claims in which the pressurising means is actuated to apply the second force to the collapsible container by depressing a button, or by removing a cap or a latch.

7. An injector assembly according to any of the preceding claims, wherein the injector assembly comprises a liquid medicament contained within the collapsible container, and wherein the first force acts to continuously pressurise the liquid medicament to a pressure that is greater than atmospheric pressure but lower than 10 bars, preferably less than 5 bars.

8. An injector assembly according to any preceding claim comprising a delivery means for delivering the liquid medicament to the patient, for example a hypodermic needle.

9. An injector assembly according to claim 8 comprising a shield for the delivery means, for example a needle shield, and a linkage system, wherein said linkage system actuated the pressurising means when the shield is retracted or removed.

10. An injector assembly according to any of the preceding claims, wherein the collapsible container is defined by rigid internal walls and a piston that is slidably arranged within the rigid internal walls, the volume of the collapsible container varying depending on the position of the piston, the pressurising means applying the first force and the second force to the piston to pressurise a liquid medicament contained within the collapsible chamber.

11. An injector assembly according to any of claims 1 to 9, wherein the collapsible container is a bellows.

12. An injector assembly according to any of the preceding claims, wherein the collapsible container is closed by a pierceable septum.

13. An injector assembly according to any of claims 1 to 11, wherein the collapsible container is closed by normally-closed valve.

14. An injector assembly according to any of the preceding claims, wherein the injector assembly is an auto-injector, for example a hand-held auto-injector.

15. An injector assembly according to any of the preceding claims, wherein the injector assembly comprises a liquid biological medicament.
